(19) United States
(12) Patent Application Publication
(10) Pub. No.: US 2003/0065289 A1
(43) Pub. Date: Apr. 3, 2003

(54) Gastrostomy tube band

(76) Inventor: Joan Clayton, Raleigh, NC (US)

Correspondence Address: JINAN GLASGOW, P O BOX 28539, RALEIGH, NC, 276118539

(21) Appl. No.: 09/682,624

(22) Filed: Sep. 28, 2001

(51) Int. Cl. A61M 5/32

(52) U.S. Cl. 604/174; 604/331

(57) ABSTRACT

An abdominal tube band for holding medical tubes attached to a patient including a substantially rectangular fabric band having dimensions that permit the band to be releasably connectable when fitted around a human body, a pocket positioned within the band for receiving medical tube ends extending from the patient's body, and fastening means for releasably securing the band in position connected to opposite ends of the band such that when the band is fitted around a human body the opposite ends of the band will overlap for engaging the fastening means and the pocket is positioned proximal to the medical tube ends, thereby providing a comfortable tube holder that is independent of any patient garment but that provides adequate support and protection of the medical tubes and their insertion region within the patient's body.

### BACKGROUND OF THE INVENTION

(1) Field of the Invention

The present invention relates generally to a band for holding medical tube(s) and, more particularly, to an abdominal band for holding gastrostomy tubes.

(2) Description of the Prior Art

Typically, where a patient requires periodic medication or fluids administration, a medical tube or tubes are inserted into the patient's skin at a predetermined location, e.g., the abdominal region for gastrostomy tube insertion; these tubes include a closed-end portion that protrudes from the insertion area into which medication or fluids may be administered by connecting various tubes into the pre-inserted tube at the protrusion or stoma.

Prior art medical tube securing devices commonly employ belt-like devices having releasable fasteners, including hook and loop type fasteners, for securing tubes such as gastrostomy tubes. However, the tube is generally pulled through the belt via a hole and is then left exposed as a protrusion from the belt-like device. This permits inadvertent movement of the exposed tube portion, thereby causing disruption of the tube at the insertion area or stoma, potentially aggravating the insertion area surrounding the tube and even causing infection and pain, or at least discomfort. Even where a pouch enclosure has been employed, as in U.S. Pat. No. 4,799,923 issued Jan. 24, 1989 to Campbell, the pouch is secured directly to the body and the tube is inserted into the pouch from holes in the band side that directly contacts the patient skin; any slippage of this type of belt-pouch will directly move the tube with respect to the body opening or stoma, causing irritation and discomfort and potentially dislodging the tube. In fact, in this invention, it is taught that the fasteners of the pouch are intended to secure the tube to the pouch in order to reduce the possibility of the tube sliding into the patient's body. This feature will promote tube movement when the patient moves with respect to the belt device, or vice versa. This clearly teaches away from the present invention protective pocket, which promotes some tube movement within the pocket, as set forth hereinbelow.

Other devices of prior art are incorporated into a garment; however, incorporating a tube securement device into a garment would likely expose the entire garment to soiling if any leakage of the tube occurs, as in U.S. Pat. No. 5,806,096 issued Sep. 15, 1998 to Pennington. Also, disruption of the tube is likely to occur when the garment moves with respect to the patient's skin and tube insertion area or stoma, which will cause patient discomfort and possibly dislocation or dislodgement of the tube, and irritation of the tube insertion area, which may lead to infection.

Thus, there remains a need for a patient band having a protective pocket for holding gastrostomy or other medical tubes that is independent of a patient's garments and includes a pouch to hold the tube end such that the tube end does not directly contact the patient's skin, and that provides releasable and adjustable connection around a patient's body proximal to the medical tube connection to the patient such that the band does not contact the entrance point of the tube into the body.

### SUMMARY OF THE INVENTION

The present invention is directed to a patient band that is independent of a patient's garments having a protective pocket for holding gastrostomy or other medical tubes in a manner such that the pouch protects the end of the tube but allows movement of the tube end with respect to the entrance point in order to reduce the movement of the tube in proximity of the entrance point into the body, and that provides releasable and adjustable connection around a patient's body proximal to the medical tube connection to the patient.

In a preferred embodiment, breathable material is used for those portions of the band that contact the patient's skin directly and a cleanable pocket material, preferably a water-resistant synthetic material, which may be a liner or coating. Preferably, the band may either be disposable or reusable, depending upon the type of material selected for construction of the band. The material used for the band body may be woven, knitted, nonwoven or a combination of fabrics.

Also in a preferred embodiment, hook and loop type fasteners are employed at the ends of the band for easy, quick and releasable attachment of the band to the patient's body.

Thus, the present invention provides a reusable or disposable medical tube band with a pocket for holding and protecting medical tubes attached to a patient's body that is releasably connectable and positionable about a patient proximal to the medical tube insertion region on the patient.

Accordingly, one aspect of the present invention is to provide an abdominal tube band for holding medical tubes attached to a patient including a substantially rectangular fabric band having dimensions that permit the band to be releasably connectable when fitted around a human body, a pocket positioned within the band for receiving medical tube ends extending from the patient's body, and fastening means connected to opposite ends of the band for releasably securing the band in position such that when the band is fitted around a human body the opposite ends of the band will overlap for engaging the fastening means and the pocket is positioned proximal to the medical tube ends, thereby providing a comfortable tube holder that is independent of any patient garment but that provides adequate support and protection of the medical tubes and their insertion region within the patient's body and aids in the isolation of movement of the tube end from the insertion region.

Another aspect of the present invention is to provide a reusable medical tube band with a pocket for holding and protecting medical tubes attached to a patient's body that is releasably connectable and positionable about a patient proximal to the medical tube insertion region on the patient.

Still another aspect of the present invention is to provide a disposable medical tube band with a pocket for holding and protecting medical tubes attached to a patient's body that is releasably connectable and positionable about a patient proximal to the medical tube insertion region on the patient.

These and other aspects of the present invention will become apparent to those skilled in the art after a reading of the following description of the preferred embodiment when considered with the drawings.

### BRIEF DESCRIPTION OF DRAWINGS

FIG. 1 is a perspective view of a patient medical belt constructed according to the present invention.

### DETAILED DESCRIPTION

In the following description, like reference characters designate like or corresponding parts throughout the several views. Also in the following description, it is to be understood that such terms as “forward”, “rearward”, “front”, “back”, “right”, “left”, “upwardly”, “downwardly”, and the like are words of convenience and are not to be construed as limiting terms.

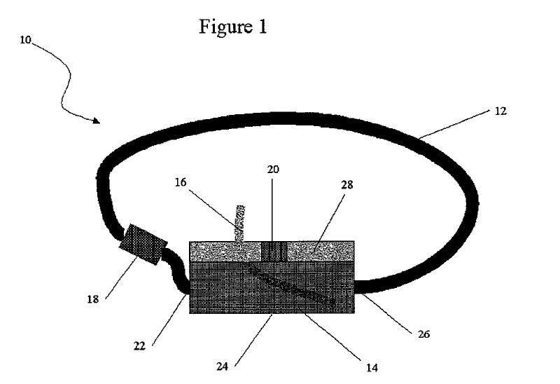

Referring now to the drawings in general, the illustrations are for the purpose of describing a preferred embodiment of the invention and are not intended to limit the invention thereto. As illustrated in FIG. 1, a preferred embodiment of the present invention is shown, generally referenced 10, including a patient band 12 having a protective pocket 14 for holding gastrostomy or other medical tubes 16 wherein the band is independent of a patient's garments and provides releasable and adjustable connection around a patient's body (not shown) by fastener means 18 for releasably securing the band proximal to the medical tube connection to the patient. The pocket is completely shielded from the patient's skin in order to provide the best protection of the tube and to prevent any dislodgement of the tube caused by movement of the patient and/or slippage or movement of the band itself. In a preferred embodiment illustrated in FIG. 1, the pocket is constructed with three closed sides 22, 24, 26 and one opening 28 that is positioned at a top side of the band for inserting the medical tube therein. The opening is large enough such that movement of the patient or the band with respect to the patient's skin will not cause movement to the tube when inserted into the protective pocket. In particular, the protective pocket permits and promotes some tube movement within the pocket such that movement of the patient with respect to the band or vice versa will not likely cause movement of the tube itself. Additionally, the pocket may include a closure 20 for additional protection and securement of the medical tube(s) when not being used to administer medication or other fluids to the patient; however, significantly, this closure is only partial in nature and is designed and configured to permit some movement of the tube with respect to the pocket and band.

In a preferred embodiment, breathable material is used for those portions of the band that contact the patient's skin directly and a cleanable pocket material, preferably a water-resistant synthetic material, which may be a liner or coating that provides a “slick” or impermeable quality such that any residual medical or fluids on the medical tube(s) will not substantially penetrate or saturate the band body material creating a leak that will be uncomfortable and potentially unsanitary condition for the patient. The interior of the pouch is preferably made of a “slick” or low-friction surface to facilitate the sliding of the tube. Also the interior of the pouch is preferably impervious to acid and other corrosive solutions that might be introduced into the tube or reflux from the tube, such as acidic gastric fluids, to facilitate cleaning and prevent leakage. Preferably, the band may either be disposable or reusable, depending upon the type of material selected for construction of the band. The material used for the band body may be woven, knitted, nonwoven or a combination of fabrics. By way of example, not limitation, plain weave sheeting material or canvas may be used for the band body.

Also, the band body material may include natural or synthetic material and/or fibers; preferably, for patient comfort, the band body material is breathable and/or wickable, in particular any of the band body material that is positioned adjacent the patient skin when attached. The band may be sized or have dimensions to accommodate a range of patient body sizes, e.g., small, medium, large, extra-large, etc. and to function effectively when applied to various areas of the body, e.g., gastrostomy tube band for application around the abdomen, arm band for tubes at the arms, wrist, elbow region, neck band for esophogeal tubes, etc. Additionally, the band and pocket may be dimensioned to receive a variety of tube sizes, which may vary with length, diameter, etc. Thus, the present invention provides either a reusable and/or disposable patient medical tube band with a pocket for holding and protecting medical tubes attached to a patient's body, depending upon the type of materials selected; cost considerations may play a role in material selection as well.

Also in a preferred embodiment, hook and loop type fasteners are employed at the ends of the band for easy, quick and releasable attachment of the band to the patient's body. Alternatively, ties, snaps, buttons, or other releasable fasteners or connectors may be used. The fasteners of any type may be positioned in various positions around the band; by way of example and as illustrated in FIG. 1, the fasteners 18 may be positioned on the side of the band in a region close to the tube insertion pocket. Alternatively, the fasteners may be positioned at the back side of the band to prevent patient removal. Basically, they may be positioned at any point or points (where multiple fasteners are desirable) along the band length that is suitable for fastening, removal, and/or removal prevention, if necessary.

Thus, the present invention provides a reusable or disposable medical tube band with a pocket for holding and protecting medical tubes attached to a patient's body that is releasably connectable and positionable about a patient proximal to the medical tube insertion region on the patient.

One preferred embodiment of the present invention includes an abdominal tube band for holding medical tubes attached to a patient having a substantially rectangular fabric band having dimensions that permit the band to be releasably connectable when fitted around a human body, a pocket positioned within the band for receiving medical tube ends extending from the patient's body, and fastening means for releasably securing the band in position connected to opposite ends of the band such that when the band is fitted around a human body the opposite ends of the band will overlap for engaging the fastening means and the pocket is positioned proximal to the medical tube ends, thereby providing a comfortable tube holder that is independent of any patient garment but that provides adequate support and protection of the medical tubes and their insertion region within the patient's body.

This preferred embodiment for the application involving a gastrostomy tube provides a band that is constructed to releasably fit around a patient's abdomen, into which a gastrostomy tube has been inserted by medical professionals. A portion of the gastrostomy tube extends externally from the patient's abdomen for removably connecting medical tubes for administering medication and fluids to a patient without having to make repeated insertions and/or injections into the patient. The band is designed and constructed to include a pocket that faces away from the band body, which is positioned adjacent to the patient's skin. The pocket provides a protective and comfortable means for preventing the gastrostomy or other medical tube from dangling freely on the outside of the patient's abdomen, openly exposed to garments and other things that can inadvertently brush against or bump into the tube, causing movement of the tube and discomfort, potentially aggravating the insertion area surrounding the tube and even causing infection and pain. Additionally, the band, in particular the lined pocket portion, prevents contact between the patient's skin and/or garments and the tube, which might irritate the patient's skin and/or stain the patient's clothing. Furthermore, advantageously, the tube is retained close to the patient's abdomen proximal the tube insertion area so that the patient will not inadvertently dislodge the tube or place pressure on the tube or stoma. Thus, use of the band according to the present invention prevents irritation, inflammation, or other disruption of the stoma, which can lead to infection. Finally, use of the band provides the patient with increased freedom of movement without increased risk of dislodging or disrupting the tube insertion area or stoma.

Also, the band may be used in either dry or wet conditions, e.g., resting or sleeping in bed, sitting, standing and/or bathing; use in all conditions helps to ensure secured and protected positioning of the tube and preventing the patient from accidentally or intentionally disrupting, dislodging or removing the tube.

Certain modifications and improvements will occur to those skilled in the art upon a reading of the foregoing description. By way of example, the device according to the present invention can also be used for many types of tubes and catheters. For example, intravenous tubes, abdominal tubes, subcutaneous tubes, tracheostomy tubes, bladder catheters, and the like can be secured by the device according to the present invention. Also, the device according to the present invention can be used for veterinary patients. All modifications and improvements have been deleted herein for the sake of conciseness and readability but are properly within the scope of the following claims.

1. An abdominal tube band for holding medical tubes attached to a patient comprising:
a substantially rectangular fabric band having dimensions that permit the band to be releasably connectable when fitted around a human body;
a protective pocket positioned within the band away from the patient's skin for receiving medical tube ends extending from the patient's body; and
fastening means for releasably securing the band in position connected to opposite ends of the band such that when the band is fitted around a human body the opposite ends of the band will overlap for engaging the fastening means and the pocket is positioned proximal to the medical tube ends,
thereby providing a comfortable tube holder that is independent of any patient garment and does not contact the stoma, but that provides adequate support and protection of the medical tubes and their insertion region within the patient's body.

2. The band according to claim 1, wherein the fastening means are hook and loop fasteners.

3. The band according to claim 1, wherein the fastening means are ties.

4. The band according to claim 1, wherein the band is formed of breathable material.

5. The band according to claim 1, wherein the band is formed of cotton.

6. The band according to claim 1, wherein the band is formed of a sheeting material.

7. The band according to claim 1, wherein the band is formed of canvas.

8. The band according to claim 1, wherein the band is formed of a material selected from the group consisting of material that is woven, knitted, nonwoven, and combinations thereof.

9. The band according to claim 1, wherein the band is disposable.

10. The band according to claim 1, wherein the band is reusable.

11. The band according to claim 1, wherein the pocket is formed of a material that is cleanable.

12. The band according to claim 1, wherein the pocket material is water resistant.

13. The band according to claim 1, wherein the pocket material is waterproof.

14. The band according to claim 1, wherein the pocket material includes a synthetic liner.

15. The band according to claim 1, wherein the pocket material includes a coated fabric.

16. The band according to claim 1, wherein the pocket material is formed from a material selected from the group consisting of nylon, polyester, polyethylene, polypropylene, and combinations thereof.

17. The band according to claim 1, wherein the pocket is closeable.

18. The band according to claim 17, wherein the pocket is closeable using hook and loop fasteners.

19. A medical band for holding medical tubes attached to a patient comprising:
a substantially rectangular fabric band having dimensions that permit the band to be releasably connectable when fitted around a human body;
a pocket positioned within the band for receiving medical tube ends extending from the patient's body; and
hook and loop fasteners for releasably securing the band in position connected to opposite ends of the band such that when the band is fitted around a human body the opposite ends of the band will overlap for engaging the hook and loop fasteners and the pocket is positioned proximal to the medical tube ends,
thereby providing a comfortable tube end holder that is independent of any patient garment and does not contact the stoma, but that provides adequate support and protection of the medical tubes and their insertion region within the patient's body.
